(19) United States
(12) Patent Application Publication
(10) Pub. No.: US 2013/0035638 A1
(43) Pub. Date: Feb. 7, 2013

(54) ANGIOPLASTY ASSEMBLY

(71) Applicant: Advanced Vascular Technology Limited, Oranmore (IE)

(72) Inventors: Paul P. Thornton, Galway (IE); Thomas J. Gaskin, Galway (IE)

(73) Assignee: ADVANCED VASCULAR TECHNOLOGY LIMITED, Oranmore (IE)

(21) Appl. No.: 13/647,787

(22) Filed: Oct. 9, 2012

Related U.S. Application Data
(63) Continuation of application No. 12/784,756, filed on May 21, 2010, now abandoned.

(30) Foreign Application Priority Data
May 22, 2009 (IE) S2009/0399

(51) Int. Cl. A61M 25/09; A61M 25/10

(52) U.S. Cl. 604/103.05; 604/96.01

(57) ABSTRACT

An angioplasty assembly having a guidewire device for use in peripheral vascular and coronary angioplasty applications. The guidewire invention has one or more angioplasty devices mounted directly on the distal tip of the guidewire. In one embodiment the angioplasty device includes an angioplasty balloon. In another embodiment, the device includes a stent balloon with expandable stent, or a self-expanding stent, or combinations of these. Preferably, the guidewire is housed within a hypotube. Optionally, the hypotube may house a retractable piercing element with a distal loop 3 suitable for piercing a vessel wall. The guidewire allows an angioplasty procedure to be completed using a single device.

### BACKGROUND OF THE INVENTION

1. Field of the Invention

The present invention relates to a medical device for use in angioplasty. The present application is a continuation of, and claims priority benefit in, U.S. nonprovisional application Ser. No. 12/784,756, filed May 21, 2010, entitled “AN ANGIOPLASTY ASSEMBLY” of the same named inventors and assigned to a common assignee, which application claims priority in Irish Patent Application No. S2009/0399 dated 22^{nd} May 2009. The entirety of those priority applications is incorporated herein by reference.

2. Description of the Prior Art

A guidewire is a wire which is intended safely and efficiently to guide another device, such as a balloon catheter, graft catheter or other device, to a target anatomical position, which may be a diagnosis or treatment site, usually in a bodily lumen or in or adjacent a bodily organ. Typically the guidewire must pass through tortuous passageways such as those of the vasculature to reach the target site. Guidewires are used in many medical diagnostic and treatment applications including percutaneous (through the skin), transluminal (inside the vessel) angioplasty (blood vessel visualisation, treatment and/or reshaping). One conventional method of treatment of blood vessel lesions, including partial or full occlusions or blockages is to perform bypass surgery where a new path is created for blood flow around the site of the blood vessel lesion. As well as in vascular applications, guidewires are also used in the treatment of other body lumens including but not limited to bile duct lesions, aneurysms and the like.

Guidewires are commonly used in minimally invasive vascular or cardiovascular procedures to guide catheters or other medical devices to a target site within the body. The guidewire is typically advanced to the site of a lesion in a vein or artery. Other interventional medical devices, such as guide catheters, therapeutic catheters, or diagnostic catheters, are introduced over or alongside the guidewire and directed to the site of the arterial or venous lesion.

Occluded or blocked vessels can occur in patients with peripheral vascular disease (PAD), and if left untreated can result in ulcerations or gangrene with associated increased risk of limb loss or even death.

The incidence of chronic peripheral vascular disease and aortic aneurysms is very high in the United States of America. Up to twelve million people have PAD in the United States, according to the American Heart Association. The incidence of PAD is expected to increase dramatically over the next five years as the age profile of the population shifts toward an increasingly ageing population. According to the American Family Physician 8% to 10% of people over 60 years of age have abdominal aortic aneurysms in the USA. The ever increasing incidence of peripheral vascular disease and aneurysms increases the need for an improved delivery system for treatment of these conditions.

Presently, angioplasty intervention requires several different instruments depending on the condition being treated and the status of the patient. Frequently the interventional clinician will make a judgement during the procedure as to the best way to proceed with the surgery, depending on what the clinician observes to be the medical need when the lesion site is accessed during the procedure. The clinician must then take a flexible approach which may require him or her to withdraw one instrument and insert a different one. In order to reduce the risk to the patient, time taken by such procedures and the associated costs, there is a need for improved instrumentation which provides the clinician with better, more flexible, less costly instrumentation.

### SUMMARY OF THE INVENTION

The present invention seeks to address the above problems. Accordingly, the present invention provides an angioplasty guidewire assembly which is flexible and which has a tip capable of opening an occluded body lumen and which has an angioplasty balloon and/or stent balloon and stent mounted thereon.

Thus, the present invention provides an angioplasty assembly comprising an elongate flexible guidewire having a distal end and a proximal end, characterised in that at least one angioplasty device is mounted on the distal end of the guidewire.

In one arrangement, the angioplasty device comprises an inflatable angioplasty balloon. In another arrangement, the guidewire includes a further angioplasty device comprising a stent balloon having a stent mounted thereon. In yet another arrangement, the angioplasty device comprises a self-expanding stent or graft. Each guidewire may include a plurality of angioplasty devices.

The angioplasty assembly ideally includes a hollow, elongate flexible hypotube having a distal end and a proximal end and at least one lumen extending along the length of the hypotube between the ends for accommodating within it the guidewire device. In a preferred arrangement, the assembly includes a handle portion connected to the proximal end of the hypotube, the handle including channel means for receiving the guidewire therethrough with the guidewire being movable within the channel along the longitudinal axis of the guidewire, the handle further including an actuator means.

In a preferred embodiment, the angioplasty assembly includes a retractable sheath member, the sheath member comprising an elongate hollow jacket having a distal end and a proximal end, the jackets being adapted to receive the guidewire within it so as to cover the or each angioplasty device mounted on the distal end of the guidewire, the jacket being connected at its proximal end to the handle actuator means so that deployment of the actuator means causes the jacket controllably to retract or advance relative to the guidewire to a desired degree to expose or enclose an angioplasty device mounted on the distal end of the guidewire.

Where the angioplasty device comprises a self-expanding stent, the jacket is disposed about the self-expanding stent to retain the stent in an unexpanded state prior to deployment of the stent at a treatment site.

According to another arrangement, the angioplasty assembly includes an elongate piercing element having a distal end and a proximal end, the piercing element being contained within a lumen of the hypotube and being movable within the hypotube along the longitudinal axis of the hypotube, the piercing element being connected to the handle actuator means and being reversibly movable within the hypotube upon deployment of the actuator means to advance the distal end of the piercing element proud of the distal end of the hypotube or to retract it into the interior of the hypotube. The distal end of the piercing element preferably has a piercing projection at its tip. The piercing element itself may comprise a wire member and the piercing projection can be formed as a loop fashioned in the wire member at its tip. Ideally, the piercing element is stiffer than the guidewire and/or hypotube so that it can be used to cross a lesion which the guidewire and/or hypotube is insufficiently stiff to cross. In such cases, if the guidewire or hypotube tip cannot be made to cross the lesion by pressure applied by the physician then the piercing tip can be advanced out of the hypotube beyond the tip of the hypotube and pressed against the lesion to cross it. In another use, the piercing element is sufficiently stiff to be able to puncture and cross through the subintimal wall of a peripheral vascular or coronary blood vessel so as to enable the lesion to be bypassed using a stent or graft.

The hypotube may have a plurality of longitudinally extending lumens. It may further include a rupturable sealing diaphragm adjacent its distal end, the distal end of the guidewire being located proximal to the diaphragm, the diaphragm being rupturable by the distal tip of the guidewire as the guidewire is advance free of the hypotube during use.

An angioplasty guidewire device or assembly of the present invention targets partially or fully occluded or blocked vessels in body lumens such as the peripheral vasculature, coronary vasculature, gall bladder ducts and other body lumens which are accessible using guidewire technology.

Further, the design has application in the treatment of peripheral vascular aneurysms.

### BRIEF DESCRIPTION OF THE DRAWINGS

The invention will now be described in more detail with reference to the accompanying drawings which show by way of example only, embodiments of an angioplasty assembly of the present invention and in which:

FIG. 1a is a schematic drawing showing in partial section the distal end of a guidewire of an angioplasty assembly according to the invention;

FIG. 1b is a view of the proximal end of the angioplasty assembly of FIG. 1a;

FIG. 2a is a sectional view of the distal end of an angioplasty assembly for use in peripheral vascular angioplasty or cardiology applications showing an angioplasty balloon and a stent balloon with stent mounted on a guidewire;

FIGS. 2b to 2d are three views illustrating the operation of the angioplasty assembly of FIG. 2a;

FIG. 2e is a view of a distal end of the guidewire element of the angioplasty assembly of FIG. 2a, in isolation from the angioplasty assembly;

FIG. 3a is a sectional view of another embodiment of an angioplasty assembly suitable for use in peripheral vascular angioplasty with a self-expanding stent mounted thereon; and

FIGS. 3b to 3d are three views illustrating the operation of the angioplasty assembly of FIG. 3a.

### DETAILED DESCRIPTION OF THE INVENTION

As used herein, the term “distal” as it refers to an angioplasty assembly or a guidewire of an assembly refers to the part of the guidewire or assembly which in use leads as the device is introduced into the patient's body and is further from the clinician deploying the device, whereas the term “proximal” refers to the other end of the guidewire or assembly which in use is nearer the clinician. “Distal” and “proximal” as they refer to body lumens have the same meaning, in terms of further from and nearer to the point of the body at which the assembly or guidewire is inserted respectively.

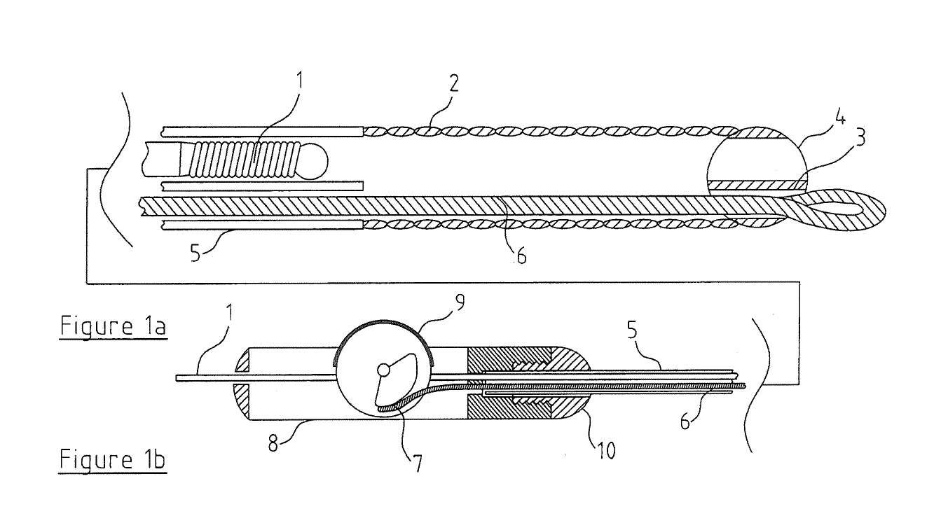

The angioplasty assembly embodiment shown in FIG. 1a has applications in angioplasty, including but not limited to peripheral vascular angioplasty. The assembly 100 includes an outer, flexible hypotube 5 constructed of a suitable material known in the art, such as Nitinol™ or stainless steel wire. Hypotube 5 is made as a wire coil 2 and is generally hollow and may have one or more longitudinal septums to divide it into compartments so that the hypotube 5 has one or more internal, longitudinally extending lumens. In the embodiment shown, the tip of the hypotube 5 is circled in FIG. 1 a to show the tip 4 of the hypotube 5 with two internal lumens defined by septum 5a. One of the lumens is occupied by a piercing element. In the embodiment shown, the piercing element comprises a wire 6 of suitable material, such as Nitinol™ having a controllable end loop 3. The other lumen is occupied by a coiled guidewire 1, such as those described in more detail below. Both the hypotube 5 and the guidewire 1 provide, alone or together, the functions of a guiding member as will be described below.

FIG. 1b is a schematic drawing showing the proximal end of the angioplasty assembly 100 of FIG. 1a. A handle 8 is provided at the proximal end and guidewire 1 is threaded through a channel of the handle so as to be movable longitudinally relative to the handle. Wire 6 is connected to an actuator, such as a push/pull wheel 9 at connection point 7 on the handle 8. Hypotube 5 is fixed to the handle 8 at the hypotube gripper 10 and guidewire 1 is threaded through the handle 8 as described and emerges at the proximal end of handle 8, where it can be manipulated by the clinician.

Angioplasty assembly 100 of FIG. 1a and FIG. 1b has use as follows, particularly in an application for clearance of one or more vessels partially or wholly blocked in the peripheral vasculature, for example in the leg of a patient. The hypotube 5 of the assembly 100 is introduced and advanced into the vasculature by known techniques and visualised by known techniques to guide it to the treatment site. The flexible hypotube 5 leads and acts as a guidewire. When the blockage site is reached, the clinician continues to advance the hypotube under a certain pressure. In some cases, this application of pressure will be sufficient to enable the tip of the hypotube 5 to cross the lesion. If not, the clinician then uses wheel 9 to deploy and advance wire 6, which up to that time had been located wholly within the hypotube 5. As wire 6 advances, its end loop 3 emerges beyond the distal tip 4 of the hypotube 5. The loop 3 is constructed to be sufficiently strong so that as it advances out of the tip 4, the loop 3 gives the physician the ability to pierce and break through the subintimal plane on the vessel, enabling the physician to circumvent the lesion in the vessel. The objective of subintimal angioplasty is deliberately to create a subintimal dissection plane beginning proximal to the occluded lesion and ending at the distal end of the lesion. Thereafter, the bridged portion of the vessel is supported by deployment of a suitable stent or graft (not shown in FIG. 1a) after which the loop 3 can be withdrawn back inside the hypotube 5 using the wheel 9. The repair stent or graft may be carried on another device contained within the hypotube 5.

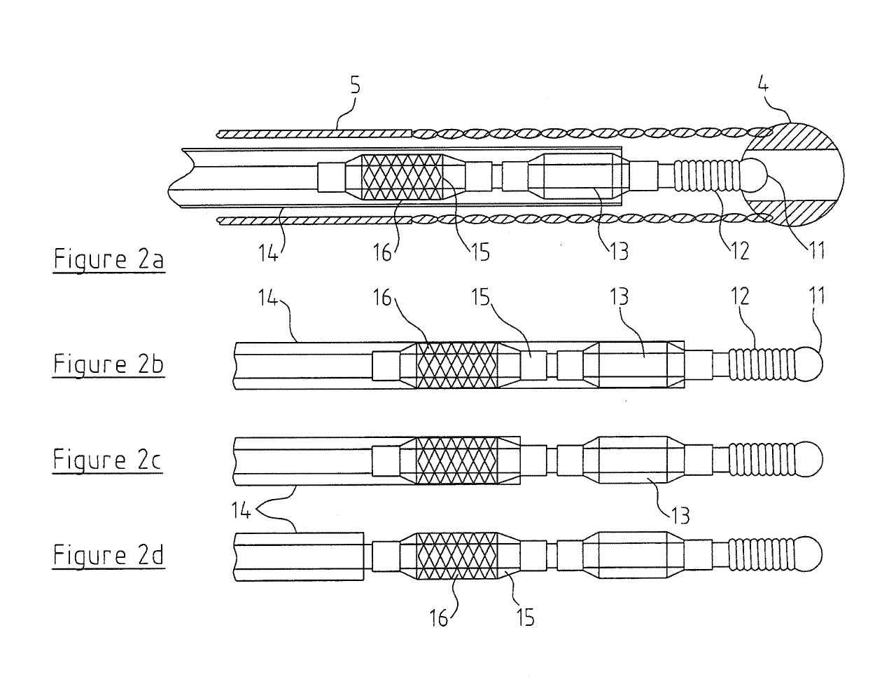

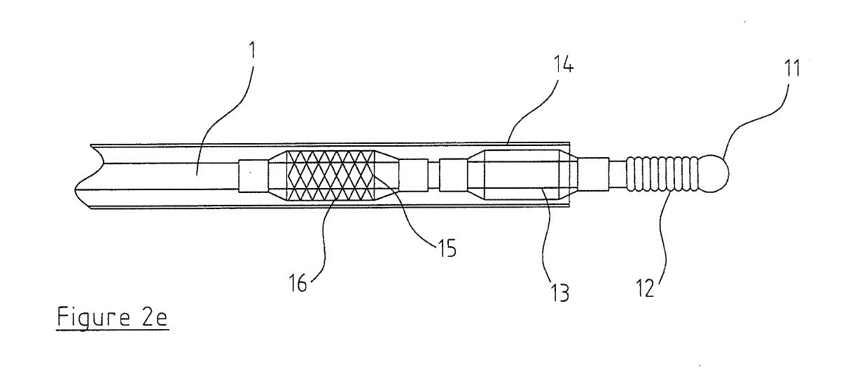

In some cases, a further lesion will exist distal to the now-repaired first encountered lesion. Using the device of FIG. 1, the clinician may go on to repair the second lesion using the existing deployed assembly 100. This is achieved by now deploying the guidewire 1 out of the distal tip 4 of the hypotube 5 and advancing it further into the vasculature until it encounters the next lesion, at which point clearance or repair of this next lesion can be attempted as will be described below in connection with FIG. 2. The clinician can do this without needing to withdraw the hypotube or guidewire as the presence of both wire 6 and guidewire 1 within the hypotube 5 provide the flexibility to conduct different procedures depending on what clinical conditions the physician encounters as the procedure is conducted.

One embodiment of the angioplasty assembly of FIG. 1 consists of a 0.035 inch (0.0889 cm) outer diameter hypotube 5, which acts as a transport device for a 0.018 inch (0.045 cm) outer diameter guidewire 1. Wire 6 has a Nitinol™ wire loop 3 that can be controlled using the wheel 9 of the handle 8 at the proximal end of the guidewire assembly.

An angioplasty assembly 200 which is suitable for use in coronary angioplasty and peripheral vascular angioplasty is shown in FIG. 2.

As shown in FIG. 2, the guidewire 1 of angioplasty assembly 200 has a first balloon 13 at the distal end of the guidewire 1 followed by, at the first balloon's proximal end, a second balloon 15 with a stent 16 crimped about its outer surface. An elongate cover sheath or jacket 14 is provided to cover balloons 13 and 15. Jacket 14 extends from an actuator wheel on a handle of the assembly and its distal end lies proximal of the tip of the hypotube. FIG. 2a illustrates schematically this embodiment of an angioplasty assembly 200 according to the present invention, with the same guidewire 1 as that of FIG. 1. The parts are similar to those described for the assembly of FIG. 1 a where the same numbers are used as appropriate. FIG. 2e shows the guidewire 1 removed from the hypotube 5.

In the case of FIG. 2a, the hypotube 5 encloses a single undivided lumen and has a tip 4. Guidewire 1 is enclosed within the lumen of the hypotube 5 and has a distal tip 11. Part of the flexible, coiled distal end 12 of guidewire 1 is shown immediately proximal of the tip 11. Proximal the guidewire tip coil 12, balloon 13 is mounted on the guidewire 1. Balloon 13 is inflatable when hypotube 5 of the guidewire assembly 200 is advanced to and in some cases across the site of a lesion. Alternatively, the guidewire 1 is advanced distally at the lesion site past the tip 4 out of the hypotube 5 toward the lesion, which typically consists of atherosclerote plaque deposits. Tip 11 of guidewire 1 is advanced so as to push it into and across the lesion. In some cases, advancement of the guidewire and inflation of balloon 13 at the lesion will be sufficient to disturb the lesion enough to clear the occluded vessel leaving it in an open condition for passage of liquid. Should that not be the case, then a supporting stent 16 may be needed to retain the vessel in the open condition and in that case the guidewire 1 may be further advanced distally so that cover jacket 14 emerges from the tip 4 of the hypotube. Cover jacket 14 covers the second stent balloon 15 with stent 16 mounted (normally by crimping) thereon. Stent balloon 15 and stent 16 are mounted on guidewire 1 proximal to balloon 13. Guidewire 1 is advanced out of jacket 14 until balloon 13 or stent balloon 15 is located at the treatment site. Ideally, this is achieved by retracting jacket 14, using an actuator such as wheel 9 shown in FIG. 1b, the proximal end of jacket 14 being connected to the wheel 9. Stent balloon 15 is inflatable to deploy and expand stent 16 at the treatment site to support the walls and retain the vessel open.

In use, the hypotube 5 will be advanced to the treatment site and as with the FIG. 1 device, can have sufficient pressure imposed upon it by the clinician so that the tip of the hypotube crosses the partially or wholly blocked lesion. Alternatively, at the lesion site, the guidewire tips may be advanced to cross the lesion. Once the lesion is crossed, the site can be treated by the clinician without need to withdraw the assembly or to introduce another device from the exterior of the patient's body, as will be described further in relation to FIGS. 2b to 2d. First, the hypotube 5 tip is retracted so that it withdraws proximal of the now-crossed lesion.

FIGS. 2b to 2d show the guidewire 1 advanced free of the hypotube 5 so that it bridges the crossed lesion. In FIG. 2b, the jacket 14 covers the balloon 13, stent balloon 15 and stent 16. In FIG. 2c, the jacket 14 is partially retracted so that the balloon 13 is free of the jacket 14 and has been inflated at the crossed lesion site to clear the occluded vessel. In FIG. 2d, the jacket 14 is still further retracted to expose stent balloon 15 which is then advanced to the cleared lesion site where it is shown inflated so as to expand stent 16 to provide scaffolding support to the treated vessel walls. A stent will not be required in all cases as the inflation of balloon 13 may be sufficient to open up and reshape the lesion adequately.

Balloons 13 and 15 are each provided with channels (not shown) running alongside the guidewire 1 for enabling an inflating liquid (usually saline) to be pumped into the interior of the balloon to inflate it.

This angioplasty assembly design of the invention eliminates the need for the use of a balloon catheter device, followed by a separate balloon catheter with a stent during percutaneous (through the skin), transluminal (inside the vessel) angioplasty (blood vessel reshaping). This provides a considerable saving in the cost of the angioplasty and the time the physician needs to perform the angioplasty, since one device could be used to complete the full treatment.

Further, the stent used in this embodiment may be a multilayered stent which has a particular application in the treatment of vascular aneurysms. A multilayered stent as used herein comprises a stent composed of a mesh, where two or more of the stents are nested, one within the other. Where the meshes have the same mesh pattern, the stents are mutually inserted with their patterns offset relative to one another. The effect is to provide a stent which retains some openings in the walls of the stent, but in which the openings are smaller than those of an individual single stent. Such a stent can be very suitable for treating an aneurysm. An aneurysm consists of a localised dilation of a blood vessel. This can occur in the brain, the aorta and in other vessels. The traditional way to treat aneurysms is surgically to open the aneurysm and replace the diseased section of the vessel with a prosthesis or artificial tube. However, these artificial tubes inhibit the passage of nutrients from the blood to the vessel wall. The multilayered stent facilitates the passage of blood nutrients to the vessel wall while at the same time, creating laminar flow in the blood vessel.

As mentioned, the angioplasty assembly 200 of FIG. 2 is suitable for use in peripheral vascular angioplasty and in coronary angioplasty. For each application, a suitably sized assembly will be chosen. For peripheral vascular angioplasty, typical outer diameter sizes for the guidewire and hypotube can be 0.018 inch (0.045 cm) and 0.035 inch (0.0889 cm) respectively. Typical outer diameters for cardiology applications for the guidewire and hypotube can be 0.014 inch (0.0225 cm) and 0.025 inch (0.063 cm) respectively.

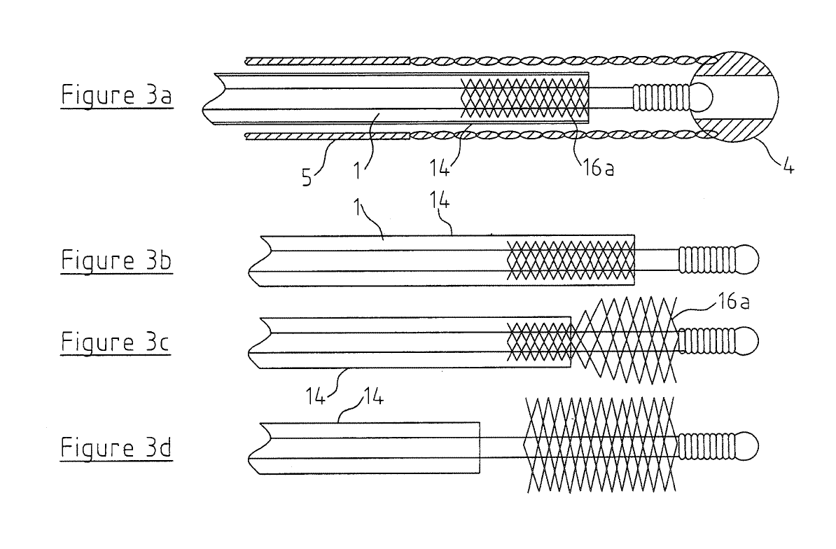

In some applications the lesion or blood vessel blockage is sufficiently soft that a balloon stent is not required to clear it. Whether or not this is the case, the reshaping of the unblocked blood vessel can be achieved or stabilised by the deployment of an expandable stent or a self expanding stent, such as a self-expanding stent made of Nitinol™. FIG. 3 shows an angioplasty assembly according to the invention with a self expanding stent 16 which is suitable for, inter alia, peripheral vascular applications. FIGS. 3a to 3d are schematic views similar to those of FIGS. 2a to 2d, but showing an embodiment of an angioplasty assembly 300 according to the invention carrying a self-expanding stent 16 mounted on a guidewire 1 housed within a hypotube 5. The self-expanding stent 16 is retained on guidewire 1 in an unexpanded state against the bias of its material by jacket 14. Jacket 14 is attached at its proximal end to an actuating means, such as a wheel, on the proximal end of the handle in similar fashion to that described and shown in respect of FIG. 1b for the wire. As the jacket 14 is retracted, stent 16 is released from within it and opens into its expanded state as it comes free of jacket 14 so that it is fully expanded when the restraining force applied by jacket 14 has been removed (FIG. 3d). Since the stent 16 is self-expanding, it does not need a balloon to expand it. A balloon (not shown) may optionally be provided distal to the stent 16 in order to accomplish the initial vessel reshaping. Prior to deploying the self-expanding stent 16, the lesion will initially have been unblocked in the manner described above; that is to say, initially the tip 4 of the hypotube and/or guidewire tip will have been advanced to cross the lesion, and if necessary, an optional balloon (not shown) near the distal tip of the guidewire 1 will have been brought to the lesion and deployed to reshape the vessel wall prior to further advancement of the guidewire 1 to position and deploy the self-expanding stent at the lesion.

Like the hypotube of FIGS. 1 and 2, that of FIG. 3 may have suitable dimension depending on the specific clinical requirements. For example, for use in adults a hypotube of outer diameter of 0.035 inch (0.0889 cm) is suitable.

Peripheral and coronary angioplasty procedures are normally carried out using a simple guidewire that does not have any other device mounted on it. The traditional guidewire is used to negotiate the anatomical tortuous path to a treatment site in the body and to cross a lesion such as a blocked vessel at the site, thereby providing a ‘train track’ upon which balloon catheters and/or stented balloon catheters and be delivered to the site of the lesion.

The angioplasty assembly according to the present invention eliminates the need for a separate balloon catheter and/or stented balloon catheter to be introduced, as it enabled the clinician to do the entire procedure using the initial introduced hypotube assembly.

Although not shown, each embodiment of the angioplasty assembly of the invention may include a diaphragm, ideally provided in the lumen of the hypotube 5 containing the guidewire 1 between the tip of the guidewire 1 and the distal tip of the hypotube 5. Such a diaphragm serves as a barrier against entry of any material (such as bodily fluid or fragments of a lesion) into the interior of that lumen, where such material could contaminate the guidewire 1. The diaphragm is made of a material which is sufficiently weak to be rupturable by the tip of the guidewire 1 when it is deployed to advance against the diaphragm.

Each of the hypotube 5, the wire 6, loop 3 and guidewire 1 may be made from any suitable, biocompatible material(s) which provide the necessary flexibility and strength characteristics to enable them to perform their intended functions. They may be made in a full range of sizes and diameters to suit their intended uses and to suit different sized vessels in adults and children.

The primary problem addressed and resolved by the angioplasty assembly of the present invention is the incorporation of multiple balloon, peripheral and cardiovascular angioplasty devices into one device; the guidewire. The technology integrates the performance capability of several peripheral angioplasty and cardiology angioplasty products into one device, which can be used flexibly for various different procedures.

The invention thus includes a guidewire which has incorporated into its body a balloon and a balloon stent that can be employed during a peripheral vascular and a cardiovascular angioplasty procedure.

The basic guidewire is of round cross section that can be manufactured in a range of diameters with a tapered distal section upon which a coil is fitted. The coil is flexible to assist the guidewire tip in negotiating through tortuous bodily lumens. The body of the guidewire incorporates a flexible guiding wire upon which is mounted a balloon or a balloon with a stent thereon or both. Another variation of the device has a self-expanding stent mounted on the guidewire, with a retractable jacket over the stent to retain it in an unexpanded state for delivery. The self-expanding stent may be fabricated from Nitinol™ or another suitable material. At the target site, the jacket is retracted allowing the stent to expand to its deployed, expanded state.

Thus the assembly of the present invention provides;
A guidewire that has peripheral angioplasty and cardiovascular angioplasty applications;
A guidewire that has a balloon mounted thereon; and/or
A guidewire that has a stented balloon mounted thereon; and/or
A guidewire that has a self expanding stent mounted thereon.

It is to be understood that the invention is not limited to the specific details described herein which are given by way of example only and that various modifications and alterations are possible without departing from the scope of the invention as defined by the appended claims.

1. An angioplasty assembly comprising an elongate flexible guidewire having a distal end and a proximal end, wherein at least one angioplasty device is mounted on the distal end of the guidewire.

2. An angioplasty assembly according to claim 1, in which the angioplasty device comprises an inflatable angioplasty balloon.

3. An angioplasty assembly according to claim 2, in which the guidewire includes a further angioplasty device comprising a stent balloon having a stent mounted thereon.

4. An angioplasty assembly according to claim 1, which includes an angioplasty device comprising a self-expanding stent or graft.

5. An angioplasty assembly according to claim 1, including a hollow, elongate flexible hypotube having a distal end and a proximal end and at least one lumen extending along the length of the hypotube between the ends for accommodating within it the guidewire device.

6. An angioplasty assembly according to claim 5, including a handle portion connected to the proximal end of the hypotube, the handle including channel means for receiving the guidewire therethrough with the guidewire being movable within the channel along the longitudinal axis of the guidewire, the handle further including an actuator means.

7. An angioplasty assembly according to claim 6, including a retractable sheath member, the sheath member comprising an elongate hollow jacket having a distal end and a proximal end, the elongate hollow jacket being adapted to receive the guidewire within it so as to cover the or each angioplasty device mounted on the distal end of the guidewire, the elongate hollow jacket being connected at its proximal end to the handle actuator means so that deployment of the actuator means causes the elongate hollow jacket controllably to retract or advance relative to the guidewire to a desired degree to expose or enclose an angioplasty device mounted on the distal end of the guidewire.

8. An angioplasty assembly according to claim 7, in which the angioplasty device comprises a self-expanding stent and the elongate hollow jacket is disposed about the self-expanding stent to retain the stent in an unexpanded state prior to deployment of the stent at a treatment site.

9. An angioplasty assembly according to claim 6, including an elongate piercing element having a distal end and a proximal end, the piercing element being contained within a lumen of the hypotube and being movable within the hypotube along the longitudinal axis of the hypotube, the piercing element being connected to the handle actuator means and being reversibly movable within the hypotube upon deployment of the actuator means to advance the distal end of the piercing element beyond the distal end of the hypotube or to retract it into the interior of the hypotube.

10. An angioplasty assembly according to claim 9, in which the distal end of the piercing element has a piercing projection at its tip.

11. An angioplasty assembly according to claim 10, in which the piercing element comprises a wire member and the piercing projection comprises a loop fashioned in the wire member at its tip.

12. An angioplasty assembly according to claim 11, in which the piercing element is capable of crossing a lesion that the guidewire cannot cross.

13. An angioplasty assembly according to claim 12, in which the piercing element is capable of puncturing the wall of a peripheral vascular or coronary blood vessel.

14. An angioplasty assembly according to claim 5, in which the hypotube has a plurality of longitudinally extending lumens.

15. (canceled)
